(19) United States
(12) Patent Application Publication
(10) Pub. No.: US 2022/0233115 A1
(43) Pub. Date: Jul. 28, 2022

(54) INFORMATION ACQUISITION DEVICE

(71) Applicant: LAPIS Semiconductor Co., Ltd., Yokohama (JP)

(72) Inventor: Noriyuki MIURA, Yokohama (JP)

(73) Assignee: LAPIS Semiconductor Co., Ltd., Yokohama (JP)

(21) Appl. No.: 17/574,529

(22) Filed: Jan. 12, 2022

(30) Foreign Application Priority Data
Jan. 25, 2021 (JP) 2021-009818

(51) Int. Cl. A61B 5/1455; A61B 5/00

(52) U.S. Cl. CPC A61B 5/14551; A61B 5/489

(57) ABSTRACT

The disclosure provides an information acquisition device capable of accurately acquiring information inside a living body as compared with a transmission type and a reflection type. The information acquisition device includes an output source that irradiates a target living body with a detection wave, and a reception part capable of receiving the detection wave irradiated to the target living body. The output source and the reception part are arranged so that an angle between a direction of irradiating the target living body with the detection wave from the output source and a direction from an irradiation point of the target living body irradiated by the detection wave to the reception part is an obtuse angle.

### CROSS-REFERENCE TO RELATED APPLICATION

This application claims the priority benefit of Japan application serial no. 2021-009818, filed on Jan. 25, 2021. The entirety of the above-mentioned patent application is hereby incorporated by reference herein and made a part of this specification.

### BACKGROUND

### Technical Field

The disclosure relates to an information acquisition device capable of non-invasively acquiring biological information by irradiating a living body with a detection wave such as light.

### Related Art

In recent years, as health consciousness increases, an information acquisition device has been developed that is used as a guideline for health condition and advice for lifestyle-related diseases by analyzing the blood vessel shape and blood components inside the living body non-invasively.

As such an information acquisition device, a reflection type (see FIG. 9) that uses a detection wave reflected from the living body as a method of irradiating the living body with a detection wave such as light and measuring biological information non-invasively and a transmission type (see FIG. 10) that uses a detection wave penetrating from the front surface of the living body to the back surface thereof are used (see, for example, Patent Literature 1).

In the detection wave of the information acquisition device for observing the inside of the living body, not only the visible light wavelength but also the near infrared wavelength (>700 nm) are used so that information deeper in the living body can be acquired.

In the information acquisition device 211 shown in FIG. 9 and the information acquisition device 312 shown in FIG. 10, in addition to determining the wavelength band in view of whether the sensitivity can be sufficiently acquired by a silicon sensor or the like, the wavelength band used as the detection wave can be determined based on the balance between the absorbance of water in the living body and the absorbance of hemoglobin in blood and red blood cells.

For the near infrared wavelength, for example, when a silicon semiconductor is used, due to the physical properties of the material thereof, for example, blood vessel information inside the living body cannot be acquired unless strong light equivalent to several watts or more is input with the output sources 220 and 320. Therefore, it is difficult to apply it to a portable device because it consumes a large amount of power, and so far, it has been limited to applications such as palm vein recognition on a relatively superficial surface.

On the other hand, recent developments of sensor devices by various companies have improved their sensitivity and performance, and even silicon semiconductors, which are advantageous in integrating functions, start to be capable of achieving high-sensitivity performance in the band exceeding 850 nm, which is a longer wavelength.

[Patent Literature 1] Japanese Patent Application Laid-open No. 2003-331272

As shown in FIG. 9, the conventional reflection type information acquisition device 211 irradiates a target living body (for example, a finger) with light from an LED as the output source 220, and detects the transmitted wave by the reception part 240, and the angle B shown in FIG. 9 is close to 0 degrees. In this conventional reflection type information acquisition device 211, the detection wave reflected from the surface of the living body is strongly reflected, and the information on the surface of the living body (for example, the fingerprint on the surface) is exaggerated and becomes noise, and it is difficult to accurately acquire the information inside the living body.

Further, as shown in FIG. 10, the conventional transmission type information acquisition device 312 irradiates a target living body (for example, a finger) with light from an LED as the output source 320, and detects the transmitted wave by the reception part 350, and the angle C shown in FIG. 10 is close to 180 degrees.

In this conventional transmission type information acquisition device 312, the distance that the detection wave travels inside the living body becomes long, and the attenuation of the detection wave is large, and it is difficult to accurately acquire the information inside the living body.

In view of the above circumstances, the disclosure provides an information acquisition device capable of accurately acquiring information inside a living body as compared with a transmission type and a reflection type.

### SUMMARY

In view of the above, an information acquisition device according to the disclosure includes an output source that irradiates a target living body with a detection wave, and a reception part capable of receiving the detection wave irradiated to the target living body. The output source and the reception part are arranged so that an angle between a direction of irradiating the target living body with the detection wave from the output source and a direction from an irradiation point of the target living body irradiated by the detection wave to the reception part is an obtuse angle.

According to the disclosure, an information acquisition device capable of accurately acquiring information inside a living body as compared with a transmission type and a reflection type can be provided.

### BRIEF DESCRIPTION OF THE DRAWINGS

FIG. 1 is a conceptual diagram of the information acquisition device according to the first embodiment.

FIG. 2 is a schematic appearance diagram showing a measurement state of the information acquisition device according to the first embodiment.

FIG. 3 is a photograph showing an example of an image acquired by the information acquisition device according to the first embodiment.

FIG. 4 is a graph showing the absorption rates of water and hemoglobin.

FIG. 5 is a graph showing an absorption spectrum of hemoglobin (oxygenated hemoglobin and deoxygenated hemoglobin).

FIG. 6 is a photograph showing an example of an image acquired by the information acquisition device according to the second embodiment.

FIG. 7 is a photograph showing an example of an image acquired by the information acquisition device according to the third embodiment.

FIG. 8 is a photograph showing an example of an image acquired by the information acquisition device according to the fourth embodiment.

FIG. 9 is a conceptual diagram of a conventional reflection type information acquisition device.

FIG. 10 is a conceptual diagram of a conventional transmission type information acquisition device.

### DESCRIPTION OF THE EMBODIMENTS

Hereinafter, an example of an embodiment of the technique of the disclosure will be described with reference to the drawings. Further, the same reference numerals are given to the same or equivalent components and parts in each drawing. In addition, the dimensional ratios in the drawings are exaggerated for convenience of explanation and may differ from the actual ratios.

### First Embodiment

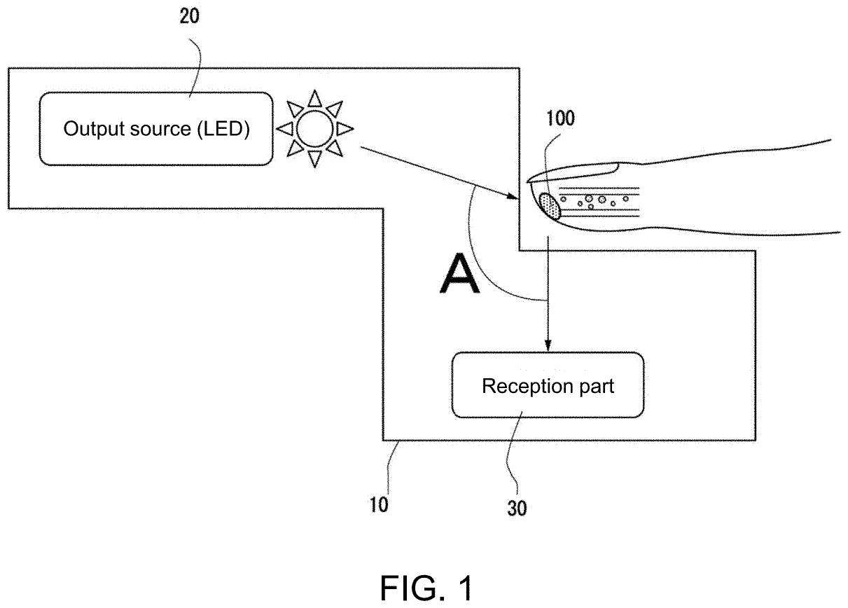

An information acquisition device 10 of the first embodiment will be described with reference to FIGS. 1 to 6.

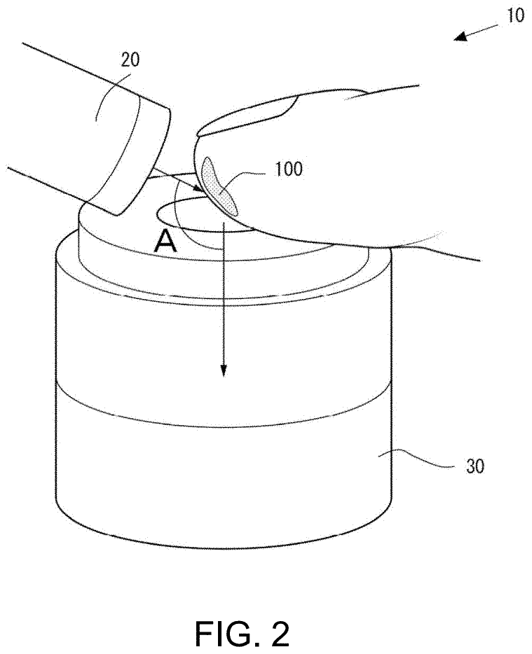

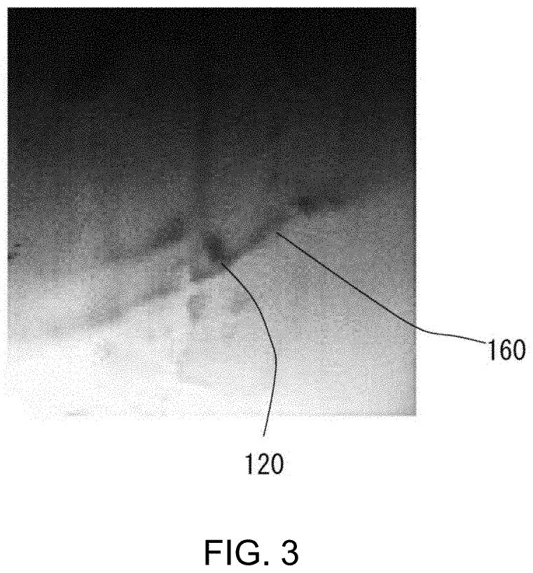

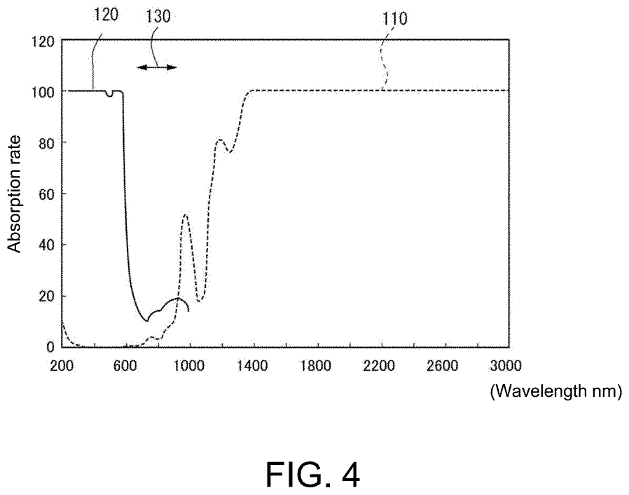

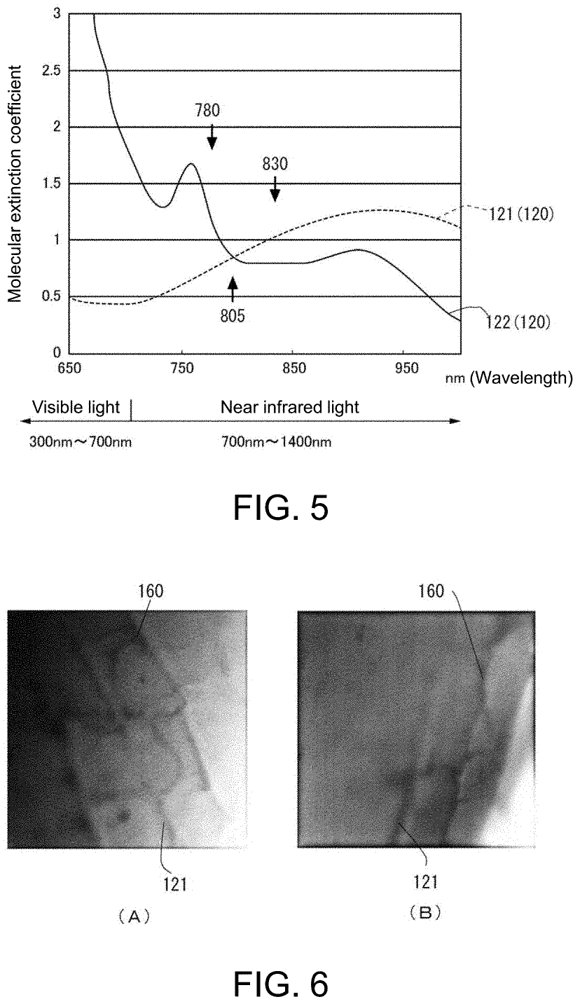

FIG. 1 is a conceptual diagram of the information acquisition device 10 according to the first embodiment. FIG. 2 is a schematic appearance diagram showing a measurement state of the information acquisition device 10 according to the first embodiment. FIG. 3 is a photograph showing an example of an image acquired by the information acquisition device 10 according to the first embodiment. FIG. 4 is a graph showing the absorption rates of water and hemoglobin. FIG. 5 is a graph showing an absorption spectrum of hemoglobin (oxygenated hemoglobin and deoxygenated hemoglobin). FIG. 6 is a photograph showing an example of an image acquired by the information acquisition device 10 according to the first embodiment.

As shown in FIG. 1, the information acquisition device 10 according to the embodiment has an output source 20 including an LED that irradiates a target living body (here, a human finger) with a detection wave, and a reception part 30 capable of receiving the detection wave irradiated to the target living body.

Though not shown in particular, the reception part 30 includes a semiconductor device having pixels including a photodiode for photodetection, and a control part that controls the detected light as a signal.

Further, though the output source 20 has an LED, a laser diode or the like may be used.

The output source 20 and the reception part 30 are arranged so that the angle (the angle A in FIG. 1) between a direction of irradiating the human finger, which is the target living body, with the detection wave from the output source 20 and a direction from an irradiation point 100 of the target living body irradiated by the detection wave to the reception part 30 is an obtuse angle (an angle greater than 90 degrees and less than 180 degrees).

In the embodiment, light, which is a detection wave, is incident from the surface of the finger of the living body, which is an object to be imaged, with respect to the light emitting direction of the detection wave from the output source 20, and the incident detection wave irradiates the irradiation point and is scattered. The reception part 30 takes an image of the scattered detection wave coming out of the finger of the living body. The irradiation point is, for example, a blood vessel (blood) of the finger. The detection wave irradiates the blood vessel and blood, is absorbed by hemoglobin or the like in the blood, is scattered, and is emitted to the outside of the living body. The output source 20 and the reception part 30 are arranged so that, with the irradiation point 100 as the apex, the angle A formed by the half straight lines extending from the apex respectively to the output source 20 and the reception part 30 is an obtuse angle.

Since the output source 20 and the reception part 30 are arranged in an obtuse-angled positional relationship, it is not necessary to pinch the finger of the living body, which is the object to be imaged, between the output source 20 and the reception part 30. Therefore, for example, as shown in FIG. 2, the information acquisition device 10 can measure a living body simply by placing a finger on the reception part 30.

Further, compared with the conventional reflection type, the detection wave reflected on the surface of the living body is not received by the reception part 30 as it is; therefore, it is not easily affected by reflection on the surface, and the amount of light that is attenuated by passing the light of the detection wave with a predetermined wavelength through the measurement site (the irradiation point) is easily reflected as the absorbance.

In addition, the distance that the detection wave travels inside the living body is shorter than that of the transmission type, and the attenuation can be reduced accordingly.

As a result, according to the embodiment, it is easier to accurately acquire the information inside the living body as compared with the transmission type and the reflection type.

Further, in the embodiment, since it is only necessary to place the finger for measurement as shown in FIG. 2, it is not necessary to insert the finger or the like of the living body deeply into a deep and dark hole for measurement, unlike the measurement in the transmission type. In the transmission type, the inserted finger is not necessarily physically pinched, pain due to application of pressure or heat is not felt, and an injection needle is not stabbed. However, for a subject who has no experience or knowledge of what is to be performed in the measurement in the transmission type, inserting a finger deeply into the measurement hole gives a psychological load such as anxiety and restraint.

In the embodiment, as shown in FIG. 2, since it is only necessary to place one's finger in a place where one can see, unlike the transmission type, it does not give the subject a psychological load, and the subject can undergo the measurement with peace of mind.

According to this embodiment, it is possible to acquire the advantages of both the reflection type and the transmission type.

Actually, in the embodiment, a photograph which imaged a blood vessel 160 inside a living body (human finger) is shown in FIG. 3. In this photograph, it is shown that while the LED of the output source 20 having a relatively low power of 7 mW is used, the part of the blood vessel 160 is blackened (dark) due to the light absorption by hemoglobin 120, and can be sufficiently identified.

Further, the embodiment is not limited to the configuration shown in FIG. 1. Specifically, for example, a convex lens may be arranged between the irradiation point 100 of the finger of the living body and the reception part 30 shown in FIG. 1. By arranging such a convex lens, the blood vessel 160 can be magnified and imaged.

Further, a non-contact thermometer capable of measuring the surface of the finger of the living body may be arranged. By measuring the temperature of the finger, it is possible to correct (calibrate) the absorbance.

Further, for the purpose of calibration, a calibration optical sensor for measuring the amount of light output from the output source 20 may be added separately.

Further, the information acquisition device 10 according to the embodiment can measure the oxygen saturation of arterial blood by simultaneously measuring the pulse of the subject by using a detection wave having a wavelength of about 800 nm, and also has a function as a pulse oximeter.

### Second Embodiment

In this embodiment, the wavelength of the detection wave output from the output source is limited to a specific wavelength from the first embodiment.

The above contents will be described in more detail below.

FIG. 4 shows the absorption rates of water 110 and hemoglobin 120, respectively.

The detection wave according to the embodiment is light having a wavelength capable of passing through a living body and having a wavelength at which the hemoglobin 120 has a higher absorbance than the water 110.

Here, the wavelength capable of passing through a living body includes a wavelength range that can easily pass through a living body, that is, a wavelength range (650 nm to 950 nm) called a “window 130 of the living body.”

The main light-absorbing substances present in the living body are the water 110 and the hemoglobin 120, which is an oxygen transport medium present in the blood, and their absorption spectra are strongly wavelength-dependent as shown in FIG. 4.

For visible light (300 nm to 700 nm), hemoglobin 120 has a large absorption rate, and the distance for which visible light can travel in the living body is short.

Further, for light having a wavelength longer than 1400 nm, water has a large absorption rate, and the distance for which the light can travel in the living body is short.

Since the absorption of the hemoglobin 120 and the water 110 is weak for the near infrared light in the wavelength range (650 nm to 950 nm) called “the window 130 of the living body,” the near infrared light in such a wavelength range can penetrate deeply into the living body. Therefore, near infrared light in such a wavelength range is often used for biopsy using light, and this wavelength range is called the “window 130 of the living body.”

FIG. 5 shows an absorption spectrum of the hemoglobin 120 (oxygenated hemoglobin 121 and deoxygenated hemoglobin 122).

The oxygenated hemoglobin 121 (indicated by a dotted line in FIG. 5) is also called oxidized hemoglobin or oxyhemoglobin HbO2, and is the hemoglobin 120 bound to oxygen, which means the state of the hemoglobin 120 in arterial blood.

The deoxygenated hemoglobin 122 (shown by a solid line in FIG. 5) is also called reduced hemoglobin or deoxyhemoglobin Hb, and is the hemoglobin 120 not bound to oxygen, and means the state of the hemoglobin 120 in venous blood.

Further, the “molecular extinction coefficient” on the vertical axis in FIG. 5 is a numerical value proportional to the absorbance when the measurement target is the same and the optical path length is the same.

Here, the “absorbance” is a light attenuation coefficient (the degree to which light is weakened according to the optical path length in a substance) calculated based on the Beer-Lambert's law, and it is used as a method for optically non-invasively estimating the amount of blood components (the oxygenated hemoglobin 121, the deoxygenated hemoglobin 122, the blood glucose level, and the like), which is important information in the living body.

In the embodiment, it is the suitable for the detection wave to be light having a wavelength at which the oxygenated hemoglobin 121 has a higher absorbance than the deoxygenated hemoglobin 122 when the artery is to be detected.

Further, it is suitable for the detection wave to be light having a wavelength at which the deoxygenated hemoglobin 122 has a higher absorbance than the oxygenated hemoglobin 121 when the vein is to be detected.

The above contents will be described in another way with the graph of FIG. 5.

A graph of the oxygenated hemoglobin 121 (shown by the dotted line in FIG. 5) showing the relationship between the wavelength of the detection wave and the molecular extinction coefficient of the detection wave in the arterial blood vessel and a graph of the deoxygenated hemoglobin 122 (shown by the solid line in FIG. 5) showing the relationship between the wavelength of the detection wave and the molecular extinction coefficient of the detection wave in the venous blood vessel intersect at a wavelength (805 nm) of the detection wave; and the wavelength of the detection wave is set between the wavelength at the intersection (805 nm) and the maximum wavelength (950 nm) or the minimum wavelength (650 nm), which is the wavelength range of the detection wave that easily passes through the living body and is called the window of the living body.

According to the embodiment, for example, when biological information of the arterial blood vessel is to be measured, it is suitable for the wavelength of the detection wave to be greater than or equal to the wavelength of the detection wave at the intersection (805 nm) of the graph (the dotted line in FIG. 5) showing the relationship between the wavelength of the detection wave and the molecular extinction coefficient of the detection wave in the arterial blood vessel and the graph (the solid line in FIG. 5) showing the relationship between the wavelength of the detection wave and the molecular extinction coefficient of the detection wave in the venous blood vessel. Further, the wavelength of the detection wave is set to be less than the maximum wavelength (950 nm) of the “window of the living body” which is the wavelength range of the detection wave that easily passes through the living body. In this way, it is possible to increase the absorbance of the arterial blood vessel in this range compared with the venous blood vessel.

As a result, the arterial blood vessel can be made blacker (darker) than the venous blood vessel in the measurement image, can be made more conspicuous, and the biological information of the arterial blood vessel can be acquired more accurately.

Further, according to the embodiment, for example, when biological information of the venous blood vessel is to be measured, it is suitable for the wavelength of the detection wave to be less than the wavelength of the detection wave at the intersection (805 nm) of the graph (the dotted line in FIG. 5) showing the relationship between the wavelength of the detection wave and the molecular extinction coefficient of the detection wave in the arterial blood vessel and the graph (the solid line in FIG. 5) showing the relationship between the wavelength of the detection wave and the molecular extinction coefficient of the detection wave in the venous blood vessel. Further, the wavelength of the detection wave is set to be greater than or equal to the minimum wavelength (650 nm) of the “window of the living body” which is the wavelength range of the detection wave that easily passes through the living body. In this way, it is possible to increase the absorbance of the venous blood vessel in this range compared with the arterial blood vessel.

As a result, the venous blood vessel can be made blacker (darker) than the arterial blood vessel in the image acquired by the received detection wave, can be made more conspicuous, and the biological information of the venous blood vessel can be acquired more accurately.

Therefore, when the artery is to be detected, it is suitable for the detection wave to be light having a wavelength of greater than or equal to 805 nm and less than 950 nm.

Further, when the vein is to be detected, it is suitable for the detection wave to be light having a wavelength of greater than or equal to 650 nm and less than 805 nm.

According to the embodiment, by setting the wavelength of the detection wave to be greater than or equal to 805 nm and less than 950 nm, the molecular extinction coefficient of the arterial blood vessel can be made greater than that of the venous blood vessel in this range (see FIG. 5).

In this way, the arterial blood vessel can be made blacker (darker) than the venous blood vessel in the image acquired by the received detection wave, can be made more conspicuous, and the biological information of the arterial blood vessel can be acquired more accurately.

According to the embodiment, by setting the wavelength of the detection wave to be greater than or equal to 650 nm and less than 805 nm, the molecular extinction coefficient of the venous blood vessel can be made greater than that of the arterial blood vessel in this range (see FIG. 5).

In this way, the venous blood vessel can be made blacker (darker) than the arterial blood vessel in the image acquired by the received detection wave, can be made more conspicuous, and the biological information of the venous blood vessel can be acquired more accurately.

FIG. 6 is a photograph showing an example of an image acquired by the information acquisition device 10 according to this embodiment. In FIG. 6, (A) is an example of imaging at a wavelength of 850 nm, and (B) is an example of imaging at a wavelength of 940 nm. In both cases, the molecular extinction coefficient of the oxygenated hemoglobin 121 is greater than the molecular extinction coefficient of the deoxygenated hemoglobin 122; therefore, the arterial blood component can be better identified, and a detailed absorption image of the blood vessel 160 inside the living body can be acquired.

### Third Embodiment

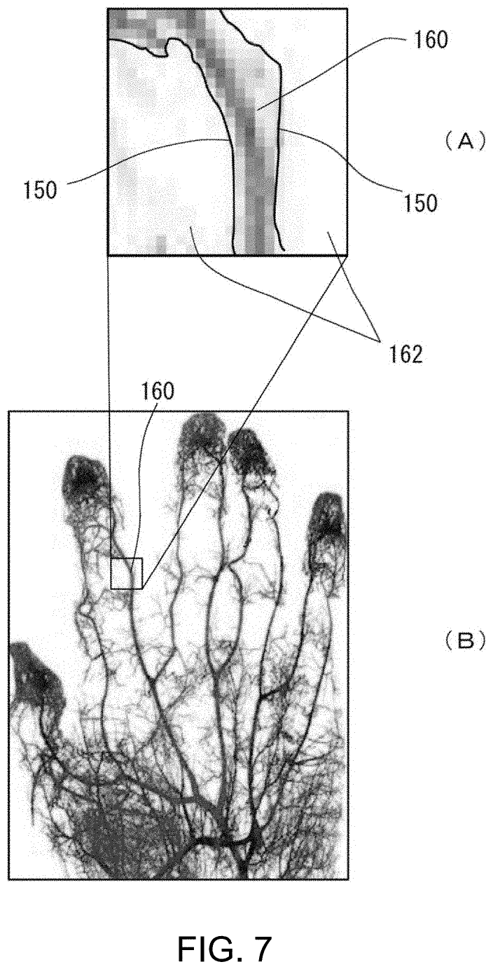

In FIG. 7, (A) is a photograph showing an example of an image acquired by the information acquisition device 10 according to the third embodiment, and (B) is a conceptual diagram showing where the image of (A) is taken.

In the embodiment, the reception part 30 receives the detection wave as an image, extracts a contour line 150 of the target portion whose contour is pixels having a pixel value difference greater than the surroundings by a threshold value or more in the received image, and calculates the absorbance only inside the contour line 150. Here, muscle and fat are formed around the blood vessel 160.

Further, the contour line 150 is created by a program built in advance in a controller inside the reception part 30 so as to connect the outer edges of the pixels with a pixel value difference greater than the surroundings by a predetermined threshold value or more.

According to the embodiment, when the absorbance is quantitatively calculated by extracting the contour line 150 of the target portion of interest, identifying the inside of the contour line 150 as the target portion, and then calculating the absorbance of only the inside thereof, for example, compared with the conventional case where information on a site other than the blood vessel 160 with hemoglobin is also taken in and measured, it is possible to acquire a more accurate value for only the target portion (for example, only the blood vessel 160).

### Fourth Embodiment

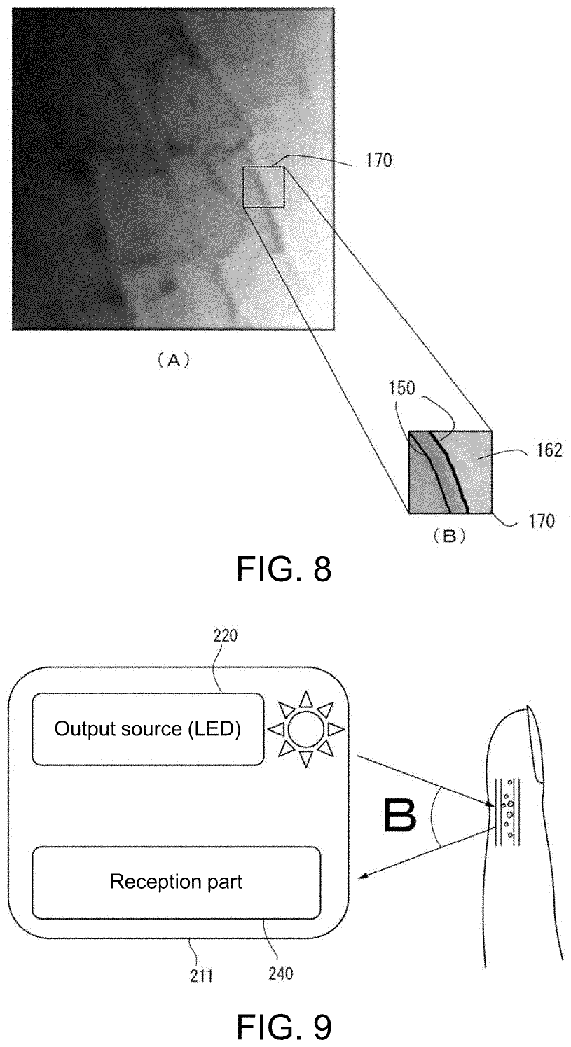

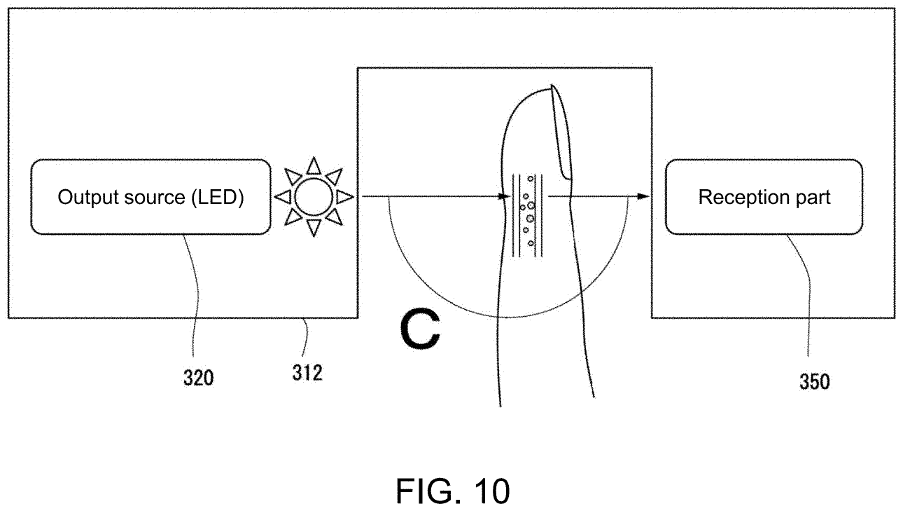

In FIG. 8, (A) is a photograph showing an example of an image acquired by the information acquisition device 10 according to the fourth embodiment, and (B) is a photograph showing an example of an image acquired by a divided pixel group 170 taken out from (A).

The reception part 30 according to the embodiment divides the image of (A) in FIG. 8 into a plurality of pixel groups 170; subtracts, in each of the divided pixel groups 170, the average value of the pixel values of the pixels of the portion not including the target portion (for example, the blood vessel 160) from the pixel values of all the pixels of the divided pixel group 170; and then calculates the absorbance inside the contour line 150. In addition, here, the portion not including the target portion (for example, the blood vessel 160) includes muscle and fat 162.

According to the embodiment, even when the output source 20 (for example, a lighting device) that outputs the detection wave does not uniformly irradiate the portion to be imaged, the influence of the uneven brightness of the background (background portion) can be suppressed, and it is possible to measure the absorbance at a more accurate quantitative value.

In the first to fourth embodiments described above, the detection wave uses light, but the detection wave is not necessarily limited to light. As long as it has a wave-like property, for example, sound is also absorbed when propagating in a substance, so ultrasonic waves or the like in a predetermined vibration band may be used similarly.

What is claimed is:

1. An information acquisition device, comprising:
an output source that irradiates a target living body with a detection wave; and
a reception part capable of receiving the detection wave irradiated to the target living body,
wherein the output source and the reception part are arranged so that an angle between a direction of irradiating the target living body with the detection wave from the output source and a direction from an irradiation point of the target living body irradiated by the detection wave to the reception part is an obtuse angle.

2. The information acquisition device according to claim 1, wherein the detection wave is light having a wavelength capable of passing through the target living body and having a wavelength at which hemoglobin has a higher absorbance than water.

3. The information acquisition device according to claim 2, wherein the detection wave is light having a wavelength at which oxygenated hemoglobin has a higher absorbance than deoxygenated hemoglobin when an artery is to be detected, and
the detection wave is light having a wavelength at which deoxygenated hemoglobin has a higher absorbance than oxygenated hemoglobin when a vein is to be detected.

4. The information acquisition device according to claim 3, wherein the detection wave is light having a wavelength of greater than or equal to 805 nm and less than 950 nm when the artery is to be detected, and
the detection wave is light having a wavelength of greater than or equal to 650 nm and less than 805 nm when the vein is to be detected.

5. The information acquisition device according to claim 1, wherein the reception part receives the detection wave as an image, extracts a contour line of a target portion whose contour is pixels having a pixel value difference greater than surroundings by a threshold value or more in the received image, and calculates an absorbance only inside the contour line.

6. The information acquisition device according to claim 2, wherein the reception part receives the detection wave as an image, extracts a contour line of a target portion whose contour is pixels having a pixel value difference greater than surroundings by a threshold value or more in the received image, and calculates an absorbance only inside the contour line.

7. The information acquisition device according to claim 3, wherein the reception part receives the detection wave as an image, extracts a contour line of a target portion whose contour is pixels having a pixel value difference greater than surroundings by a threshold value or more in the received image, and calculates an absorbance only inside the contour line.

8. The information acquisition device according to claim 4, wherein the reception part receives the detection wave as an image, extracts a contour line of a target portion whose contour is pixels having a pixel value difference greater than surroundings by a threshold value or more in the received image, and calculates an absorbance only inside the contour line.

9. The information acquisition device according to claim 5, wherein the reception part receives the detection wave as an image, divides the received image into a plurality of pixel groups, subtracts, in each of the plurality of divided pixel groups, an average value of pixel values of pixels of a portion not including the target portion from pixel values of all pixels of the plurality of pixel groups, and then calculates an absorbance inside the contour line.

10. The information acquisition device according to claim 6, wherein the reception part receives the detection wave as an image, divides the received image into a plurality of pixel groups, subtracts, in each of the plurality of divided pixel groups, an average value of pixel values of pixels of a portion not including the target portion from pixel values of all pixels of the plurality of pixel groups, and then calculates an absorbance inside the contour line.

11. The information acquisition device according to claim 7, wherein the reception part receives the detection wave as an image, divides the received image into a plurality of pixel groups, subtracts, in each of the plurality of divided pixel groups, an average value of pixel values of pixels of a portion not including the target portion from pixel values of all pixels of the plurality of pixel groups, and then calculates an absorbance inside the contour line.

12. The information acquisition device according to claim 8, wherein the reception part receives the detection wave as an image, divides the received image into a plurality of pixel groups, subtracts, in each of the plurality of divided pixel groups, an average value of pixel values of pixels of a portion not including the target portion from pixel values of all pixels of the plurality of pixel groups, and then calculates an absorbance inside the contour line.
